(19) United States
(12) Patent Application Publication
(10) Pub. No.: US 2016/0120821 A1
(43) Pub. Date: May 5, 2016

(54) DRUG ELUTING SILICONE GEL SHEETING FOR WOUND HEALING AND SCAR REDUCTION

(71) Applicant: Allergan, Inc., Irvine, CA (US)

(72) Inventors: Jordan Thompson, Chino Hills, CA (US); Alexei Goraltchouk, Rensselaer, NY (US)

(21) Appl. No.: 14/987,373

(22) Filed: Jan. 4, 2016

Related U.S. Application Data
(62) Division of application No. 14/080,580, filed on Nov. 14, 2013.
(60) Provisional application No. 61/729,980, filed on Nov. 26, 2012.

(51) Int. Cl. A61K 9/70; B29C 35/02; B29C 41/50; A61K 31/355; A61K 9/00

(52) U.S. Cl. CPC A61K 9/7007; A61K 31/355; A61K 9/0014; B29C 41/50; B29C 35/02; B29K 2083/00

(57) ABSTRACT

Provided are methods of making devices that can be applied to the skin to promote healing of wounds and the reduction or prevention of scarring. The devices include compositions of various silicone compounds loaded with active pharmaceutical agents.

This application is a divisional of U.S. Patent Application Ser. No. 14/080,580 filed on Nov. 14, 2013, which application claims priority to U.S. Provisional Patent Application No. 61/729,980, filed Nov. 26, 2012, the entire disclosure of each of these applications being incorporated herein by this reference.

The present invention generally pertains to a medical device, and more specifically relates to a drug eluting silicone gel sheeting and process for promoting skin wound healing and/or the prevention of scarring.

### BACKGROUND

The first step of the wound healing process is inflammation, which triggers the formation of fibrous tissue, or scar tissue, by proliferation of fibroblasts, mucopolysaccharides, and glycosaminoglycans at the wound site. Scarring is thus a natural part of the healing process. With the exception of very minor lesions, significant skin wounds following accidents, disease, or surgery all result in some degree of visible scarring. Scarring in many instances results in a loss of flexibility, diminished nerve response, and possibly a loss of range of motion. Scarring has obvious cosmetic problems, particularly on the face and hands. Visible scarring may occur as a result of incisions made in breast reduction surgery, C-sections, abdominoplasties, excision of scars, abrasions, accidental cuts and as a result of burns.

Naturally, new products are continually being developed and marketed to address wound healing and scar reduction. Conventional products include gel sheets and gel ointments, for example, silicone-based gel sheets and ointments. The use of these silicone-based polymers and gels has been proposed for reducing the appearance of scars when used consistently for sufficiently extended periods of time. Specific examples of such commercial products include Neosporin Scar Solutions™ sheets, Cica-Care™ sheets, Mepiform™ scar dressings, and Dermatix™ silicone gel.

Gunter, et al., U.S. Pat. No. 7,683,234, discloses devices, bandages, kits and methods for controlling or regulating the mechanical environment of a wound to ameliorate scar and/or keloid formation. The devices are configured to be removably secured to a skin surface in proximity to the wound site and shield the wound from endogenous and/or exogenous stress. The entire disclosure of this patent is incorporated herein by this specific reference.

Eede, et al. U.S. Patent Application Publication No. 2007/0093161, discloses silicone products for treatment of skin, in the form of a dressing or sheet and comprising silicone gels and including adhesive surface and an elastic material comprising polyamide or polyester, and UV protection features.

There still remains a need for improved devices for promoting wound healing and/or scar reduction.

### SUMMARY

Provided are compositions of various silicone compounds loaded with active pharmaceutical agents (API), which are suitable for promoting the healing of skin wounds and the reduction or prevention of scarring.

In one aspect of the invention, a device suitable for applying to the skin to promote healing of a wound in the skin or reduction of a scar on the skin, is provided, wherein the device generally comprises a silicone gel sheet comprising silicone and having a first surface or side, and a substantially opposing second surface or side; and a drug, or active agent, mixed with the silicone, the active agent being effective to promote wound healing and/or scar reduction when in contact with a wound or scar. In one embodiment, the active agent is present in the sheet in a concentration gradient generally decreasing from the first surface to the second surface. The drug may be present, for example, in a higher concentration at the skin contacting surface, decreasing in gradient through the thickness of the device.

In one aspect of the invention, a flexible wound covering is provided, the wound covering generally comprising a silicone gel sheet, for example, a cured silicone gel material, containing an active agent effective to promote wound healing or reduce scar formation when placed in contact with a wound, the active agent dispersed in, for example, mixed with the silicone and being present in the sheet in a concentration gradient generally decreasing from a first side of the sheet to a second side of the sheet opposing the first side of the sheet. The silicone may be a cured silicone.

The active agent may be selected from a pharmaceutical compound, a protein, a vitamin or other beneficial agent. In some embodiments, the active agent is a vitamin, including but not limited to: Vitamin A, Vitamin C, Vitamin E, Vitamin D, and Vitamin K. In some embodiments, the agent is an healing or antibiotic, anti-infective agent, or other agent that is known to be beneficial when applied topically to a wound, for example, a laceration, scratch, cut, abrasion, incision, or burn wound. Such agents may include, for example, known beneficial topical formulations including, but not limited to: silver sulfadiazine, sulfacetamide sodium, sulfacetamide sodium/sulfur, retapamulin, erythromycin bacitracin/neomycin/polymyxin, bacitracin/polymyxin, bacitracin/neomycin/polymyxin b/pramoxine, bacitracin, gentamicin, lidocaine/neomycin/polymyxin, mafenide, neomycin/polymyxin b/pramoxine, neomycin/polymyxin, neomycin, sulfacetamide sodium/sulfur/urea, and sulfacetamide sodium/urea.

In some embodiments, the device further comprises an adhesive, for example, disposed on the first side of the gel sheet, for promoting adherence of the sheet to a wound or scar. The adhesive may be selected from the group consisting of polyacrylate-based, polyisobutylene-based, and silicone-based pressure sensitive adhesives.

In other embodiments, the device further comprises a carrier effective to enhance permeability of the active agent into skin.

In another aspect of the invention, methods of making a device suitable for applying to skin to promote healing of a wound in the skin or reduction of a scar on the skin are provided. A method of the invention may comprise the steps of providing an uncured silicone gel, applying the uncured silicone gel to a mold surface to form an uncured silicone gel sheet having an exposed first surface and a substantially opposing second surface in contact with the mold surface, and contacting the first surface of the uncured silicone gel sheet with an active agent/solvent solution. The solvent allows the active agent to penetrate the silicone and become at least partially encompassed or encapsulated thereby.

The method may further comprise step of applying a vacuum to the silicone gel sheet. The vacuum may be applied to the second surface, the surface of the gel sheet opposing the surface on which the active agent/solvent was applied, to pull the agent into the gel. The step of applying a vacuum causes the drug or other active agent to be drawn into the silicone material. The method further may include curing the silicone gel sheet having the active agent/solvent. Curing may be performed during or after the vacuum step. The method is effective to form a silicone-gel sheet including an active agent present in a concentration gradient decreasing from the first surface to the second surface.

In some embodiments, the method further comprises the step of swelling the silicone gel in a solvent for the gel prior to or during the step of applying the active agent/solvent solution. Swelling the gel in a solvent may further enhance the ability of the agent applied thereto to penetrate into the silicone sheet and/or become incorporated into the sheet.

Each and every feature described herein, and each and every combination of two or more of such features, is included within the scope of the present invention provided that the features included in such a combination are not mutually inconsistent.

### DETAILED DESCRIPTION

The present invention provides a simple and effective device for promoting wound healing and/or reduction of scar formation and methods for making the same.

In some embodiments of the invention, the device comprises a silicone gel in the form of a sheet, and an active agent or drug contained in or dispersed through the silicone gel. The agent is releasable from the gel when the gel is placed in contact with the skin.

Active agents, in accordance with the invention, are agents that are beneficial to treating or promoting wound healing or scar reduction. The agents used within the scope of the invention include, for example, any known beneficial agents, for example, one or more antioxidants, analgesics, anesthetics, anti-arthritis drugs, anti-inflammatory drugs, anti-migraine drugs, cardiovascular medications, pain-killers, anti-smoking drugs, natural or synthetic hormones, anti-hypertension agents, anti-depressants, antibiotics, anti-cancer agents, antiemetics, anti-infectants, contraceptives, diabetes drugs, steroids, anti-allergy agents, anti-migraine agents, dieting agents, vitamins, minerals, herbs, nutritional supplements, or the like. Agents that are known to increase cell growth or vascularization or reduce inflammation may also be considered and are considered to be within the scope of the invention. Such agents include, for example, platelet derived growth factor, epidermal growth factor, anti-inflammatory drugs, VEGF, Fibroblast growth factor.

The active agent may be uniformly dispersed throughout the gel sheet. In other embodiments, the active agent or drug is more concentrated in certain regions, for example, on a surface of the gel sheet to be in contact with the skin. In one aspect of the invention, a silicone gel sheet is provided which delivers a beneficial drug at a higher concentration when the sheet is initially applied to the skin. For example, the device includes a silicone gel sheet having an active agent present in a concentration gradient, rather than being uniformly concentrated throughout the gel sheet. In some embodiments, the active agent is concentrated, for example, has a relatively high concentration, near a surface of the silicone gel sheet, for example, on a surface or side of the silicone gel sheet that will be in contact with the wound or scar. The concentration decreases incrementally or substantially continually, in a direction away from the highly concentrated surface, for example, in a direction perpendicular to the surfaces of the sheet.

The process for drug-loading can include mixing the active into the gel before casting the gel. In some embodiments, multiple gel layers are formed, the layers partially cured, each layer having a different drug concentration. The formed, partially cured layers are then assembled by placing the layers together, one on the other, to form a composite sheet assembly having a drug gradient defined by the differing concentrations of adjacent layers. Curing can be done after the assembly step, to cause the layers to fuse together. This produces a drug eluting silicone gel sheet having a drug concentration gradient. In some instances the gel is swollen with an aqueous solution or solvent for the gel, to promote better penetration of the active agent into the silicone.

In other embodiments, a solution containing a drug is applied to the top of a gel sheet before or after curing. Such sheets can be used individually or can be layered together, for example, as described above.

The present silicone gel sheet may provide an effective means for reducing appearance of a scar, or at least decreasing worsening of a fresh scar, reducing wound healing time and decreasing scar formation.

In general, within the scope of the current invention a silicone gel is poured into a mold, the drug is loaded into the gel, and the gel is cured. In one embodiment of the invention, the drug is first mixed with the gel before the gel is cast such that the drug is dispersed evenly throughout the gel.

In yet another embodiment of the invention, the gel is first cast and allowed to dry by vacuum. Then a drug/solvent solution is cast onto the dried gel for a set amount of time. The solvent allows the drug to penetrate the top portion of the gel while the majority of the drug remains on the surface. In another embodiment of the invention the gel is first cast, dried, and cured. Then the cured silicone gel sheet is placed in a drug/solvent solution and allowed to swell and is gently to facilitate better uptake. After a set amount of time, the gel is removed from the solution and allowed to dry in vacuum. The drug is then dispersed fully throughout the gel. The drug loaded silicone gel will then be applied directly to a wound site in order to accelerate the healing process and improve scar appearance. Within this embodiment of the invention the drug solution can be applied in a multitude of ways, including spraying the drug solution onto the surface or dip coating the gel sheet, for example.

Within the scope of this invention the drug solution that is added to the silicone gel can also contain a carrier aspect. The carrier includes inactive or active ingredients that are used to enhance the permeability of the drug through the skin at the wound site to make the drug more effective. General classes of permeability enhancing carriers include terpenes, liposomes, azones, lipids, and alcohols. Specific permeability enhancers include dimethyl sulphoxide (DMSO), 1-[2-(decylthio)ethyl]azacyclopentan-2-one (HPE-101), Labrafac CC, Labrafil, Labrasol, Transcutol, isopropyl myristate, oleic acid, propylene glycol, ethanol, and azone. Other carrier types could include stabilizers. Stabilizers allow higher than normal amount of drug to be kept in the silicone gel and also allow for specific loading of the drug depending on where the stabilizing carrier is added to the gel. Stabilizers can also allow for controlled release of the drug from the silicone gel to create more favorable release kinetics in order to achieve an improved therapeutic effect.

In another embodiment of the invention a silicone sheet is first cast into a mold at a set thickness, for example about 1 mm. The silicone sheet is allowed to dry and then the silicone gel is cast over the silicone sheet such that the silicone gel completely covers the silicone sheet. The silicone gel is then allowed to dry. The drug is then loaded into the silicone gel by casting the drug/solvent solution on top of the silicone gel and the solvent is allowed to evaporate. The sample is then cured resulting in a drug loaded silicone gel sheet backed by a silicone sheet. The silicone sheeting backing provides support and improved mechanical properties to the silicone gel sheet. This results in a more durable product and allows for a thinner product which would be advantages to patients concealing the product under their clothing. The silicone sheet backing will also hinder patients from putting the “wrong side” of the product onto their skin if the drug is loaded onto only one side.

In another embodiment of the invention, the same process is followed but the drug is loaded into the silicone gel sheet by mixing it in at a set concentration before the silicone gel sheet is cast onto the silicone sheet. Examples of possible silicone sheets include both RTVs and HTVs including MED 1511, MED 4850, MED 4714, MED 6640, MED 6600, and MED 6400, all available from NuSil Technology.

Different types of silicone sheets can have a larger effect on the overall product. For example different durometer silicone sheet backings will change the durability and the softness of the overall product. MED 6640 and MED 6600 are HTV phenyl silicones. The phenyl in their backbone will reduce the drugs ability to diffuse through the silicone backing. Alternatively, a fluorinated silicone could be used to further reduce the drugs ability to diffuse through the silicone backing. The thickness of the silicone sheet can be altered in order to achieve different properties of the overall product. For instance if a high durometer silicone is used, for example, MED 4850, then the silicone backing could be made thinner to achieve the desired softness while reducing the overall thickness of the product and still maintaining necessary mechanical properties for durability. Increasing the thickness of the silicone sheet, in general, will improve mechanical properties and durability, while making the final product stiffer.

In another embodiment of the invention the silicone gel sheet is cast onto a mesh or fabric that is more aesthetically appealing and less adhesive then a silicone sheet. The mesh also serves to reinforce the silicone gel sheet to make it more durable and give it better mechanical properties.

In one embodiment of the invention the finished drug loaded silicone gel sheet is packaged into a thermoform with release liner on either side and sterilized. The sample is then applied directly to a wound or scar for up to 1 week, up to 2 weeks, up to 3 weeks, or more, for at least about 1 hour, about 2 hours, about 3 hours, about 4 hours, about 5 hours, about 6 hours, about 7 hours, about 8 hours, about 9 hours or about 10 hours each day, resulting in improved wound healing and scar reduction or scar reduction. Conventional non-drug loaded silicone gel sheets may thereafter be applied to the improved wound or scar for another period of time, for example, for example, about 1 additional week to about 9 additional weeks.

Within the scope of the invention the silicone gel layer can be varied to achieve different results. A thinner gel is typically easier to handle and conceal under clothing while a thicker gel results in better mechanical properties, durability, and the potential to load more drug into the gel.

The thickness of the sheet of the invention can vary from between about 0.1 mm up to about 20 mm, depending on what properties are needed. In some embodiments the sheeting has a thickness in a range of between about 1.0 mm to about 10 mm, about 2 mm to about 9 mm, about 3 mm to about 8 mm, about 4 mm to about 7 mm, about 5 mm to about 6 mm. A thin gel can be reinforced with a silicone sheet backing or a mesh or fabric backing to give it stronger mechanical properties and better durability. Different durometer silicone gels can be used as well in order to achieve different levels of softness and tackiness. Generally the lower durometer, or softer, the silicone gel the more tacky it becomes. The tackiness is necessary for adherence to the wound site.

Once the silicone gel sheet has been cured a release liner is added onto both sides of the gel before the gel is packaged into a thermoform and sterilized. In one embodiment of the invention, the release liner is placed in the mold before the gel is cast such that the gel forms with the release liner already attached (the release liner would be on the non-wound applied side). The release liner on the wound side can be added before or the gel is cured. Example release liners include PTFE, Teflon, polypropylene, HDPE, LDPE, or any other release liner known in the art.

Factors that can be varied in the current invention in order to achieve different results include: the type of silicone gel used, the drug that is loaded into the silicone gel, the thickness of the silicone gel, the solvent used in casting the gel, the solvent used in loading the drug, the process by which the solvent is removed from the drug/solvent mixture, the time at which the drug is added to the silicone gel (i.e. how “dry” the gel is), the concentration of drug loaded into the gel, and the drug loading process. Different silicone gels will have different mechanical properties which could alter the effectiveness of the treatment, the drug that is loaded into the silicone gel will change the final outcome based on its properties (i.e. whether it decreases healing time, decreases the chance of infection, reduces inflammation, reduces scar tissue formation, etc.). Some examples of silicone gels that can be used are MED3-6320, MED-6340, MED-6345, MED-6350 from Nusil Technology. The thickness of the silicone gel will affect how soft the gel feels and the amount of drug loaded into it (depending on the drug loading process). The solvent used in casting the gel will vary the working time for how long it takes the gel to dry, which could alter how much drug is incorporated into the material and how much remains on the surface. The solvent used in loading the drug into the gel and the process by which the solvent is removed will determine how long the drug/solvent solution has to permeate into the gel which will affect the overall distribution of the drug throughout the gel. The concentration of drug loaded into the gel will affect how efficacious the treatment is, and possibly the release profile of the drug. The time at which the drug is added to the silicone gel and the drug loading process will vary the release profile of the drug. If the drug is mixed with the silicone gel before it is cast or added to the gel by swelling the gel then the release profile could be more linear in comparison to a drug that is applied to the top of the gel. A drug that is applied to the top of the gel in a drug/solvent solution would have some integration into the top part of the gel which would have some sustained release, but a majority of the drug would remain directly on the surface.

### EXAMPLE

A silicone gel is prepared by mixing a dispersion of 30% part A in Xylene and a dispersion 30% part B in Xylene of MED 6345, a clear, tacky silicone gel available from NuSil Technology, at a ratio of 1:1. A drug solution, for example, an aqueous solution containing Vitamin E, is then added to the mixture at a specific concentration and the whole dispersion is then allowed to mix by rolling for 16 hours. The aqueous solution may contain other components as well, for example, one or more permeability enhancers, for example, dimethyl sulphoxide. The dispersion is then cast into a flat rectangular mold. Due to the drug and gel being mixed homogenously together, the drug is dispersed fully throughout the gel. The gel is then placed in an oven at 125° C. for 2 hours to cure. The sheet is removed from the mold and cut into segments having sizes and shapes useful to cover wounds.

### EXAMPLE

A silicone gel is prepared by mixing a dispersion of 40% part A in Xylene and a dispersion 40% part B in Xylene of MED 6350, available from NuSil Technology, at a ratio of 1:1. The dispersion is then cast into a flat rectangular mold. The gel is allowed to dry for an hour in vacuum. Once dry, the mold is removed from vacuum and a drug/solvent solution, for example, an anti-infective agent mixed with a solvent for the silicone gel, is poured directly onto the top of the gel. The mold is placed in vacuum for an hour so that all the solvent is removed and the drug is pulled into the gel by the vacuum. The gel is then placed in an oven at 125° C. for 2 hours to cure. The result is a silicone gel sheet with a majority of the drug loaded onto the surface of one side of the gel and a gradient of the drug concentration decreasing away from the one side. The sheet is then cut into segments having sizes and shapes suitable for use as a wound dressing to promote healing and/or scar reduction. The side of the sheet having the highest concentration of drug may be placed in contact with the wound.

Although the invention has been described with a certain degree of particularity, it is understood that the present disclosure has been made only by way of example, and that numerous changes in the combination and arrangement of parts can be resorted to by those skilled in the art without departing from the scope of the invention, as hereinafter claimed.

What is claimed is:

1. A method of making a device suitable for applying to skin to promote healing of a wound in the skin or reduction of a scar on the skin, the method comprising:
providing an uncured silicone gel;
applying the uncured silicone gel to a mold surface to form an uncured silicone gel sheet having an exposed first surface and a substantially opposing second surface in contact with the mold surface;
contacting the first surface of the uncured silicone gel sheet with an active agent/solvent solution;
applying a vacuum to the silicone gel sheet;
curing the silicone gel sheet having the active agent/solvent to form a silicone-gel sheet including an active agent present in a concentration gradient decreasing from the first surface to the second surface.

2. The method of claim 1 wherein the active agent is selected from a pharmaceutical compound, a protein, a vitamin and combinations thereof.

3. The method of claim 1 further comprising the step of applying an adhesive to the first surface of the silicone gel sheet.

4. The method of claim 3 wherein the adhesive is selected from the group consisting of polyacrylate-based, polyisobutylene-based, and silicone-based pressure sensitive adhesives.

5. The method of claim 1 further comprising the step of swelling the silicone gel in a solvent for the gel prior to or during the step of applying the active agent/solvent solution.

6. The method of claim 1 wherein the active agent is a vitamin.

7. The method of claim 1 wherein the active agent is Vitamin E.

8. The method of claim 1 wherein the active agent is an antibiotic.
